(19) United States
(12) Patent Application Publication
(10) Pub. No.: US 2013/0053534 A1
(43) Pub. Date: Feb. 28, 2013

(54) PROCESS FOR PREPARING NEOPENTYL GLYCOL

(71) Applicants: Helmut Kronemayer, Heidelberg (DE); Kai Stehmeier, Mannheim (DE); Ralf Edinger, Kaiserslautern (DE); Stefan Rittinger, Mannheim (DE); Michael Steiniger, Neustadt (DE)

(72) Inventors: Helmut Kronemayer, Heidelberg (DE); Kai Stehmeier, Mannheim (DE); Ralf Edinger, Kaiserslautern (DE); Stefan Rittinger, Mannheim (DE); Michael Steiniger, Neustadt (DE)

(73) Assignee: BASF SE, Ludwigshafen (DE)

(21) Appl. No.: 13/588,661

(22) Filed: Aug. 17, 2012

Related U.S. Application Data
(60) Provisional application No. 61/526,278, filed on Aug. 23, 2011.

(51) Int. Cl. C07C 29/141; B01D 3/34; C08G 63/02; B01D 3/14

(52) U.S. Cl. 528/272; 203/87; 203/50; 568/862

(57) ABSTRACT

A process for distilling an aqueous NPG mixture comprising NPG, a tertiary amine, water and the adduct of tertiary amine and formic acid (amine formate), said distillation being performed in a distillation column, which comprises drawing off a gaseous stream in the upper region of the column and feeding it to two condensers connected in series, the first condenser being operated in such a way that a portion of the gaseous stream is condensed in the first condenser and the second condenser being operated in such a way that the uncondensed portion of the gaseous stream is essentially fully condensed in the second condenser, the condensed stream from the first condenser being recycled fully or partly as reflux into the column.

The present application incorporates by reference Provisional U.S. application No. 61/526,278 which was filed Aug. 23, 2011.

The present invention relates to a process for distilling an aqueous neopentyl glycol (NPG) mixture. The present application further provides a process for preparing NPG, and the further conversion of the NPG thus obtained to polyester resins, unsaturated polyester resins, lubricants or plasticizers.

Neopentyl glycol is used as a raw material for the production of saturated and unsaturated polyester resins, for lubricants, for plasticizers, for powder coatings and for glass fiber-reinforced polymers.

Neopentyl glycol is generally prepared in a two-stage process, in which isobutyraldehyde (IBA) is first reacted with formaldehyde (FA) in an aldol addition to form hydroxypivalaldehyde (HPA), which can be hydrogenated directly in a second process stage to give neopentyl glycol (NPG).

An overview of a two-stage process for preparing NPG can be found, for example, in WO-A1-2010079187.

WO 2010066674 also discloses a two-stage process for preparing NPG and the purification of the NPG prepared. WO 2010066674 describes a process for distilling an aqueous polymethylol mixture comprising an NPG, a tertiary amine, water and the adduct of tertiary amine and formic acid (amine formate), which comprises performing the distillation in a distillation column connected at the bottom to an evaporator, the bottom temperature being above the evaporation temperature of the monoester of formic acid and NPG (NPG formate) which forms during the distillation. In a preferred embodiment, the condenser is generally operated at a temperature at which the predominant portion of the low boilers is condensed at the appropriate top pressure. In general, the operating temperature of the condenser is in the range from 0 to 80° C., preferably 20 to 50° C. The condensate obtained in the condenser is preferably recycled into the distillation column to an extent of more than 30% by weight, preferably to an extent of more than 60% by weight. The condensate is preferably recycled into the top of the column. In the condenser, the condensate obtained is a mixture of low boilers which is fed to the column as described above, predominantly as reflux. For example, the low boiler mixer obtained in the condenser may comprise amine, water and isobutanol from isobutyraldehyde and methanol from formaldehyde.

In the context of the present invention, it has been found that a portion of the condensate has to be discharged in order to prevent low boilers from accumulating in the column. More particularly, the accumulation of amine formate and/or formic acid leads to increased formation of NPG formate, as a result of which the yield of NPG is reduced. Together with the condensate discharged, a relevant amount of NPG and/or NPG formate is therefore generally also discharged from the process. In an industrial scale process, the amount of NPG and/or NPG formate discharged may be a few hundred to several thousand tonnes per year. This means not only a loss of NPG yield but also requires a higher level of complexity in the disposal of the amounts of distillate discharged.

It was an object of the present invention to provide a process for purifying an aqueous NPG mixture which has only low losses of NPG and in which only small amounts of NPG and/or NPG formate need be discharged. Thus, the yield of NPG which can be purified by means of the process according to the invention should be increased. In addition, the amounts of discharge streams to be purified and to be disposed of should be kept as small as possible.

The object of the present invention was achieved by a process for distilling an aqueous NPG mixture comprising NPG, a tertiary amine, water and the adduct of tertiary amine and formic acid (amine formate), said distillation being performed in a distillation column, which comprises drawing off a gaseous stream in the upper region of the column and feeding it to two condensers connected in series, the first condenser being operated in such a way that a portion of the gaseous stream is condensed in the first condenser and the second condenser being operated in such a way that the uncondensed portion of the gaseous stream is essentially fully condensed in the second condenser, the condensed stream from the first condenser being recycled fully or partly as reflux into the column.

The aqueous NPG mixture used in the process preferably has the following composition:
20 to 90% by weight of NPG,
0 to 10% by weight of methanol,
0 to 5% by weight of tertiary amine,
0 to 5% by weight of organic secondary compounds,
0.01 to 5% by weight of the adduct of tertiary amine and formic acid (amine formate),
remainder water.

The aqueous NPG mixture more preferably has the following composition:
50 to 80% by weight of NPG,
0.1 to 5% by weight of methanol,
0.01 to 5% by weight of tertiary amine,
0 to 5% by weight of organic secondary compounds,
0.01 to 5% by weight of the adduct of tertiary amine and formic acid (amine formate),
remainder water.

Such aqueous NPG mixtures are preferably obtained by multistage reaction of isobutyraldehyde with formaldehyde. In this case, an aqueous hydroxypivalaldehyde (HPA) mixture is first prepared from formaldehyde and isobutyraldehyde in an aldol reaction in a first stage. In a second stage, the aqueous HPA mixture thus obtained can be hydrogenated directly with hydrogen to give an aqueous NPG mixture in the hydrogenation process. However, it is also possible to react the HPA mixture with further formaldehyde in a Cannizzarro reaction to give an aqueous NPG mixture. Preferably, however, the aqueous NPG mixture is obtained by the hydrogenation process.

An aqueous HPA solution is prepared preferably by reacting isobutyraldehyde with formaldehyde in the presence of a tertiary amine.

Formaldehyde is generally used in the process as an aqueous formaldehyde solution. Industrially available formaldehyde is sold typically in aqueous solution in concentrations of 30, 37 and 49% by weight. However, it is also possible to use formaldehyde solutions of up to 60% by weight in the process.

Industrial formaldehyde generally comprises formic acid as a result of the preparation. The degradation products of formic acid can reduce the service life of the hydrogenation catalyst in the downstream hydrogenation stage, which can result in a decrease in the yield of NPG. In a particular embodiment, formaldehyde which has a formic acid content of 150 ppm or less is used. Such formaldehyde can be obtained, as described in the application WO 2008107333, by treatment of formaldehyde or of an aqueous formaldehyde solution with basic ion exchangers. Useful anion exchangers include ion exchangers which are known per se, are strongly basic, weakly basic or moderately basic, and are in gel form or macroporous form. These are, for example, anion exchangers of the polystyrene resin structure crosslinked with divinylbenzene, having tertiary amino groups as functional groups. Also useful are ion exchangers based on acrylic acid or methacrylic acid crosslinked with divinylbenzene, or resins prepared by condensation of formaldehyde and phenol. Specific useful examples include the commercial products Ambersep® 900, Amberlyst® and Amberlite® from Rohm and Haas, Philadelphia, USA, and Lewatit® from Lanxess, Leverkusen.

A further starting material used in the aldolization is isobutyraldehyde.

The preparation of isobutyraldehyde is described, for example, in the chapter “Butanals” (Ullmann's Encyclopedia of Industrial Chemistry, Published Online: 15 Sep. 2000, DOI: 10.1002/14356007.a04_{—}447). It can be prepared, for example, by hydroformylation of propylene.

The purity of the isobutyraldehyde used is preferably more than 95% by weight, more preferably more than 97% by weight and more preferably more than 99% by weight.

The tertiary amines used may be amines as described, for example, in DE-A 28 13 201 and DE-A 27 02 582. Particular preference is given to tri-n-alkylamines, especially triethylamine, tri-n-propylamine, tri-n-butylamine and trimethylamine. Very particular preference is given to trimethylamine (“TMA”), triethylamine (“TEA”) and tri-n-propylamine (“TPA”), since these compounds generally have a lower boiling point than NPG and hence distillative removal from the reaction mixture is facilitated. Particular preference is given to using trimethylamine (“TMA”) as the tertiary amine in the reaction.

The aldol reaction (first stage) can be performed with or without addition of organic solvents or solubilizers. The use of solvents which form suitable low-boiling azeotropic mixtures with the low-boiling compounds in the individual distillations of the process according to the invention can possibly lower the energy expenditure in these distillations and/or facilitate the distillative removal of the low boilers from the high-boiling compounds. Suitable solvents are, for example, cyclic and acyclic ethers, such as THF, dioxane, methyl tert-butyl ether, or alcohols such as methanol, ethanol or 2-ethylhexanol.

In the aldol reaction, the molar ratio of isobutyraldehyde added in fresh form in each case to the amount of formaldehyde added is appropriately in the range from 1:1 to 1:5, preferably in the range from 1:1.01 to 1:3.5 and more preferably in the range from 1:1.02 to 1:1.5 and especially preferably in the range from 1:1.03 to 1:1.1.

The amount of tertiary amine catalyst added in the aldol reaction is, in relation to the isobutyraldehyde added, generally 0.001 to 0.2, preferably 0.01 to 0.07, equivalent, which means that the amine is typically used in catalytic amounts.

The aldol reaction is performed generally at a temperature of 5 to 100° C., preferably of 15 to 80° C. The reactions described for the aldol reaction can be performed at a pressure of generally 1 to 30 bar, preferably 1 to 15 bar, more preferably 1 to 5 bar, appropriately under the autogenous pressure of the reaction system in question.

The aldol reaction can be performed batchwise or continuously. The aldol reaction is preferably performed in a continuous stirred tank reactor or a continuous stirred tank cascade. To adjust the residence time, a portion of the reaction output from one stirred tank can be recycled into the particular stirred tank reactor.

The overall residence time of the first stage (aldolization) is preferably 0.25 to 12 hours, more preferably 0.5 to 8 hours and especially preferably 1 to 3 hours. In a preferred embodiment, the residence time in the individual reactors of a reactor cascade is preferably selected such that the overall residence time is divided equally between the individual reactors.

The output from the aldol reaction comprises typically unconverted starting compounds, such as formaldehyde, alkanals, and also the tertiary amine catalyst used and possibly water. The output additionally comprises hydroxypivalaldehyde (HPA). The output typically also comprises impurities and by-products from the aldol reaction, such as formic acid, which can form as a result of Cannizzaro or Tishchenko reaction from formaldehyde, such as HPN, and formate salts of the amine catalysts used, such as trimethylammonium formate. The output from the aldolization comprises preferably 40 to 80% by weight of HPA and more preferably 50 to 70% by weight of HPA.

In a preferred embodiment, the output from the aldolization has the following composition:
HPA: 40 to 80% by weight;
water: 10 to 50% by weight;
IBA: 0 to 20% by weight;
FA: 0 to 10% by weight;
tert. amine: 0 to 10% by weight.

In a further preferred embodiment, the output from the aldolization has the following composition:
HPA: 50 to 70% by weight;
water: 15 to 40% by weight;
IBA: 1 to 10% by weight;
FA: 0.5 to 5% by weight;
tert. amine: 0.5 to 5% by weight.

The output from the aldol reaction is subsequently separated, typically by distillation. This involves feeding the output from the aldol reaction to a distillation apparatus, generally a column, in which it is separated into more and less volatile constituents. The distillation conditions are generally selected such that a fraction of low boilers is formed, in which the essential components present are unconverted alkanal, with or without water, formaldehyde and methanol. In the case of use of trimethylamine (TMA) as the tertiary amine, the distillation conditions are selected such that TMA is also partly present in the low boiler fraction and to a small degree in the bottom product. In the case of use of triethylamine (TEA), the distillation conditions are selected such that TEA is enriched in the bottom product. This low boiler fraction can be recycled into the first stage of the hydrogenation process, the aldol reaction, or sent to a further workup stage. Removal of the low boiler fraction leaves, after the distillative workup outlined, a relatively nonvolatile bottom product consisting essentially of HPA, water, formic acid and amine formate, which can be used as the HPA-comprising stream in the process according to the invention.

However, it is also possible to use an HPA-comprising stream which has been prepared by other prior art processes, for example by the processes known from WO 01/51438, WO 97/17313 and WO 98/29374.

The HPA content in the output of the aldol reaction after removal of the low boiler fraction in a customary HPA-comprising stream is, according to the disclosures cited above, within a wide range from 20 to 95% by weight, preferably from 40 to 85% by weight and more preferably from 50 to 80% by weight.

The HPA-comprising stream from the aldolization comprises, as well as HPA, generally additionally water and further different organic compounds, for example unconverted reactants or by-products of the aldolization. Examples of different organic compounds are acetals, hemiacetals, methanol, esters, amine formate, etc. The water is supplied to the reaction system generally via the metered addition of the formaldehyde, since formaldehyde is generally used as an aqueous solution.

Preferably, the HPA-comprising stream from the aldolization which is used in the process according to the invention comprises less than 10% by weight of NPG, more preferably less than 5% by weight of NPG and especially preferably less than 3% by weight of NPG, based on the HPA-comprising stream. In a preferred embodiment, the output of the aldol reaction after removal of the low boiler fraction does not comprise any additional organic solvent, in order not to dilute the concentration of the HPA in the HPA-comprising stream. This is because a high concentration of HPA in the hydrogenation feed enables smaller dimensions of the hydrogenation reactor and the use of smaller amounts of catalyst, as a result of which the overall capital and operating costs can be reduced.

Preferably, the composition of the HPA-comprising stream from the aldolization after removal of the low boiler fraction, which is used in the process according to the invention, is:
50 to 85% by weight of HPA;
15 to 50% by weight of water;
remainder: other organic compounds,
and more preferably
60 to 80% by weight of HPA;
20 to 40% by weight of water;
remainder: other organic compounds.

The output from the aldolization can, as described above, be hydrogenated in a second stage with hydrogen to give NPG (hydrogenation process), or it can be reacted with formaldehyde in the presence of strong bases in a Canizzarro reaction to given NPG.

Preferably, the output from the aldolization is hydrogenated.

In the hydrogenation, preference is given to using catalysts which comprise at least one metal of transition groups 8 to 12 of the Periodic Table of the Elements, such as Fe, Ru, Os, Co, Rh, Ir, Ni, Pd, Pt, Cu, Ag, An, Zn, Cd, Hg, preferably Fe, Co, Ni, Cu, Ru, Pd, Pt, more preferably Cu, preferably on a support material. The support material used is preferably a support material composed of the oxides of titanium, of zirconium, of hafnium, of silicon and/or of aluminum. The usable catalysts can be prepared by processes known from the prior art for preparing such supported catalysts. Preference may also be given to using supported catalysts which comprise copper on an aluminum oxide- or titanium dioxide-containing support material in the presence or absence of one or more of the elements magnesium, barium, zinc or chromium. Such catalysts and preparation thereof are known from WO 99/44974. In addition, supported copper catalysts as described, for example, in WO 95/32171 and the catalysts disclosed in EP-A 44 444 and DE 19 57 591 are suitable for the hydrogenation.

The hydrogenation can be performed batchwise or continuously, for example in a reactor tube filled with a catalyst bed, in which the reaction solution is passed over the catalyst bed, for example in trickle or liquid phase mode, as described in DE-A 19 41 633 or DE-A 20 40 501. It may be advantageous to recycle a substream of the reaction output, optionally with cooling, and to pass it through the fixed catalyst bed again. It may equally be advantageous to perform the hydrogenation in a plurality of reactors connected in series, for example in 2 to 4 reactors, in which case the hydrogenation reaction in the individual reactors upstream of the last reactor is performed only up to a partial conversion of, for example, 50 to 98%, and only in the last reactor is the hydrogenation completed. It may be appropriate to cool the hydrogenation output from the preceding reactor before its entry into the next reactor, for example by means of cooling apparatus or by injecting cold gases, such as hydrogen or nitrogen, or introducing a substream of cold reaction solution.

The hydrogenation temperature is generally between 50 and 180° C., preferably 90 and 140° C. The hydrogenation pressure employed is generally 10 to 250 bar, preferably 20 to 120 bar.

The hydrogenation feed is generally mixed with tertiary amine upstream of the hydrogenation reactor input until the hydrogenation output has a pH of 7 to 9. It is also possible to feed the hydrogenation feed and the tertiary amine separately into the reactor and to mix them therein. The tertiary amines used may be the aforementioned tertiary amines, especially TMA.

In a particularly preferred embodiment, the hydrogenation is performed according to the teaching of PCT/EP2011/057538, the contents of which are explicitly incorporated by reference. This application discloses a process for preparing neopentyl glycol (NPG) by continuously hydrogenating hydroxypivalaldehyde (HPA) with hydrogen in the liquid phase in the presence of a hydrogenation catalyst in a hydrogenation reactor, by combining an HPA-comprising stream with an NPG-comprising stream to given a hydrogenation feed and introducing the hydrogenation feed into the hydrogenation reactor and additionally supplying at least one pH regulator selected from the group consisting of tertiary amine, an inorganic base, an inorganic acid and an organic acid, to the HPA-comprising stream or to the NPG-comprising stream or to the hydrogenation feed in order to establish a pH of 7.0 to 9.0 at the outlet of the hydrogenation reactor, wherein the weight ratio of HPA to NPG in the hydrogenation feed is in the range from 1:100 to 50:100 and the proportion of HPA and NPG in the hydrogenation feed is at least 50% by weight, based on the hydrogenation feed, and in the case that the pH regulator is supplied to the HPA-comprising stream either the HPA-comprising stream comprises less than 50% by weight of HPA or the residence time between the supply of the pH regulator and the combination of the NPG-comprising stream with the HPA-comprising stream is less than 5 minutes or the temperature of the HPA-comprising stream is less than 75° C.

The reaction output from the hydrogenation is an aqueous NPG mixture comprising NPG, a tertiary amine, water and the adduct of tertiary amine and formic acid (amine formate).

As mentioned above, the aqueous polymethylol mixture preferably has the following composition:
50 to 85% by weight of NPG,
0 to 10% by weight of methanol,
0 to 5% by weight of tertiary amine,
0 to 5% by weight of organic secondary compounds,
0.001 to 5% by weight of the adduct of tertiary amine and formic acid (amine formate),
15 to 50% by weight of water.

The aqueous NPG mixture more preferably has the following composition:
60 to 80% by weight of NPG,
0.1 to 5% by weight of methanol,
0.01 to 5% by weight of tertiary amine,
0 to 5% by weight of organic secondary compounds,
0.01 to 5% by weight of the adduct of tertiary amine and formic acid (amine formate),
20 to 40% by weight of water.

The organic secondary compound present may, for example, be isobutanol.

The aqueous NPG mixture is purified by distillation, by removing low boilers from NPG. According to the invention, the low boilers are removed from the aqueous NPG mixture by a process for distilling an aqueous NPG mixture comprising NPG, a tertiary amine, water and the adduct of tertiary amine and formic acid (amine formate), said distillation being performed in a distillation column, which comprises drawing off a gaseous stream in the upper region of the column and feeding it to two condensers connected in series, the first condenser being operated in such a way that a portion of the gaseous stream is condensed in the first condenser and the second condenser being operated such that the uncondensed portion of the gaseous stream is essentially fully condensed in the second condenser, the condensed stream from the first condenser being recycled fully or partly as reflux into the column.

The distillation is preferably performed in such a way that low boilers, such as water, isobutanol, methanol, formic acid, amine formate and tertiary amine, are drawn off under reduced pressure as a gaseous stream from the upper region of the column, especially when the amine used has a lower boiling point than the NPG formed, as is the case for TMA, TEA and TPA. When a tertiary amine having a higher boiling point than the NPG formed is used, the tertiary amine is removed together with the NPG formed at the bottom and enriched in the column bottom in a downstream distillation stage, while NPG is drawn off as the top product.

Typically, a portion of the amine formates reacts during the distillation in the column bottom or in the stripping section of the column with NPG to form the free amines and the formates of NPG. This preferably forms the monoester of formic acid and NPG, which is referred to in the context of this disclosure as NPG formate. The amines released by the transesterification reaction are generally removed in the distillation together with the other low boilers at the top of the column. The distillation should therefore preferably be regulated such that the concentration of the NPG formates formed in the bottom output is kept low and the target product, the NPG, is of maximum purity. This is preferably done by selecting, in the distillation, a bottom temperature above the evaporation temperature of the NPG formate, such that the NPG formates are completely or very substantially completely converted to the gas phase by evaporation. The improvement in the yield and in the product quality brought about by this measure is probably attributable to the fact that the NPG formates typically have higher boiling points than the other low boilers, and a portion of the NPG formates is therefore generally already precipitated in the rectifying section of the columns at an appropriate reflux ratio. The NPG formates precipitated in the rectifying section can hydrolyze with water to reform formic acid and NPG. The formic acid is typically removed at the top of the column, while NPG can generally be discharged from the column bottom.

The distillation column preferably has internals for increasing the separating performance. The distillative internals may, for example, be present as a structured packing, for example as a sheet metal packing such as Mellapak 250 Y or Montz Pak, B1-250 type. It is also possible for a structured packing with relatively low or increased specific surface area to be present, or it is possible to use a fabric packing or a structured packing with another geometry, such as Mellapak 252Y. Advantages in the case of use of these distillative internals are the low pressure drop and the low specific liquid holdup compared to, for example, valve trays. The internals may be present in one or more sections.

The output from the hydrogenation is preferably fed in within a spatial region between ¼ and ¾ of the theoretical plates of the distillation column, more preferably in a spatial region between ⅓ and ⅔ of the theoretical plates of the distillation column. For example, the feed may be somewhat above the middle of the theoretical plates (ratio 3:4). The number of theoretical plates is generally in the range from 5 to 30, preferably 10 to 20.

The top pressure of the column is more preferably 0.001 to 0.9 bar, more preferably 0.01 to 0.5 bar. On the industrial scale, the vacuum is typically obtained by means of a steam ejector. In the column bottom, preference is given to establishing a temperature which is above the evaporation temperature of the NPG formate, such that the NPG formate is converted completely or very substantially completely to the gas phase.

Particular preference is given to establishing a temperature which is 5% to 50% above the boiling temperature of the NPG formate and most preferably 10% to 20% above the boiling temperature of the NPG formate at the particular column top pressure.

For example, in the case of preparation of NPG using TMA as the tertiary amine and a pressure at the top of the column of 175 mbar, a column bottom temperature of preferably 150 to 170° C., more preferably of 160 to 165° C., can be established. The energy required for the establishment of the bottom temperature and for the evaporation of the low boilers out of the bottoms is typically introduced by means of an evaporator in the column bottom. The evaporator is typically a natural circulation evaporator or forced circulation evaporator. However, it is also possible to use evaporators with a short residence time, falling film evaporators, helical tube evaporators, wiped film evaporators or a short path evaporator. The evaporator can be supplied with heat in a suitable manner, for example with 16 bar steam or heat carrier oil.

According to the invention, a gaseous stream is drawn off in the upper region of the column, preferably in the range from 2/3 to 3/3, most preferably in the range from 3/4 to 4/4, of the theoretical plates (counted from the bottom), and especially preferably at the top of the column. The gaseous stream preferably comprises amine, water, isobutanol, methanol and NPG formate which has not been precipitated in the rectifying section of the column.

The composition of the gaseous stream is preferably:
NPG formate: 0 to 20%
water: 50 to 95%
methanol: 0 to 20%
amine: 0 to 10%
isobutanol: 0 to 10%
remainder: organic compounds

The composition of the gaseous stream is more preferably:
NPG formate: 0.001-5%
water: 80 to 90%
methanol: 5 to 15%
amine: 0 to 5%
isobutanol: 0 to 5%
remainder: organic compounds

Organic compounds present in the gaseous stream drawn off generally comprise formic acid, which originates from the hydrolysis of NPG formate, and/or amine formate.

According to the invention, the gaseous stream from the upper region of the columns is supplied to two condensers connected in series.

The first condenser is operated in such a way that a portion of the gaseous stream is condensed. Preferably 5 to 90% by weight, more preferably 10 to 80% by weight, even more preferably 20 to 70% by weight and especially preferably 25 to 50% by weight of the gaseous stream is condensed out in the first condenser. According to the invention, the stream condensed in the first condenser is recycled fully or partly into the column as reflux. The inventive partial condensation can achieve discharge of low boilers, such as formic acid and amine formates, while simultaneously reducing the discharge of NPG and/or NPG formate via the top. This is enabled by the fact that the NPG formate formed is predominantly condensed out in the first condenser, can be hydrolyzed in the condensate vessel with water to give formic acid and NPG, and is introduced into the column as reflux. The NPG can then be recovered here as the product of value via the bottom, while the formic acid evaporates, is condensed only partly in the first condenser and is discharged from the column together with the other low boilers after condensation in the second condenser.

The amount of condensate is preferably such that a reflux can be recycled into the column such that sufficient separating action is achieved. The reflux is preferably adjusted such that the predominant amount of the NPG or NPG formate is retained in the column. The reflux is therefore preferably adjusted such that the temperature at the point where the gaseous stream is drawn off is below the evaporation temperature of NPG formate at the particular column pressure and above the evaporation temperature of the low boilers to be removed, such as water, methanol, isobutanol and amine. The column temperature in the region of the draw point of the gaseous stream is therefore preferably in the range from 0.2× boiling point of NPG formate to 0.8× boiling point of NPG formate, more preferably in the range from 0.3× boiling point of NPG formate to 0.6× boiling point of NPG formate, and especially preferably in the range from 0.35× boiling point of NPG formate to 0.5× boiling point of NPG formate.

Too high a reflux flow rate can lead to the effect that the bottoms become too cold since the evaporation energy of the reboiler is no longer sufficient to evaporate the low boilers. Due to the higher concentration of low boilers, the purity of the NPG drawn off at the bottom can decrease. Too low a reflux flow rate can lead to the effect that the temperature in the region of the top of the column rises to an excessive degree, such that a greater amount of NPG is drawn off with the gaseous stream.

Therefore, in general, the reflux rate should preferably be selected such that the temperature in the bottom of the column and in the region of the draw point of the gaseous stream is within the aforementioned preferred ranges.

In general, for this purpose, 10 to 50% by weight, more preferably 10 to 40% by weight and most preferably 20 to 40% by weight of the gaseous stream drawn off is recycled into the column as reflux. To achieve better separation performance and hence a higher purity of the NPG-containing bottoms output from the column, the reflux rate and hence the amount condensed out can optionally, however, as described above, also be increased or reduced. The exact operating conditions of the column can be determined in accordance with the separation performance of the column used by the person skilled in the art on the basis of the known vapor pressures and evaporation equilibria of the components supplied and to be separated by conventional calculation methods in a routine manner.

The reflux rate can be regulated by the throughput of cooling medium through the condenser. Thus, the reduction in the amount of coolant supplied can reduce the proportion of the amount of the gaseous stream drawn off which is condensed out. Preferably, the condensed stream from the first condenser is recycled completely into the column as reflux. Since the condensation temperature of the first condenser is generally sufficiently high that only a low proportion of the low boilers is condensed out, the accumulation of low boilers in the column can be very substantially avoided.

For this purpose, it is preferable that the first condenser is operated at a temperature which is 1 to 80%, more preferably 1 to 20% and most preferably 1 to 10% below the condensation temperature of water at the appropriate top pressure. The cooling medium used here may preferably be water or a coolant mixture, for example glycol/water. Preferably, as described below, the coolant which originates from the outlet of the second condenser is passed as coolant into the inlet of the first condenser.

In a particular embodiment, the top pressure of the column is in the range from 0.01 to 0.5 bar. At 0.01 bar, the condensation temperature of water is approx. 8° C.; at 0.5 bar, the condensation temperature of water is approx. 82° C. The result of this is that the temperature of the cooling medium at the inlet of the first condenser is preferably 0.01 bar in the range from 8° C.×0.2 to 8° C.×0.99, more preferably in the range from 8° C.×0.8 to 8° C.×0.99 and especially preferably in the range from 8° C.×0.9 to 8° C.×0.99. A further result is that the temperature of the cooling medium at the inlet of the first condenser at 0.5 bar is in the range from 82° C.×0.2 to 82° C.×0.99, more preferably in the range from 82° C.×0.8 to 82° C.×0.99 and especially preferably in the range from 82° C.×0.9 to 82° C.×0.99.

The condenser may be configured from virtually all condensers known to those skilled in the art, for example plate condensers, shell and tube condensers or coil condensers. The condenser is preferably configured as a shell and tube condenser. The condenser may be operated vertically or horizontally; the condensation may take place in the shell space or in the tubes. In a preferred vertical variant, in the case of gas supply from above in a particularly preferred variant, a liquid distributor can distribute a portion of the already condensed liquid homogeneously in the tubes, and thus reduce or prevent solid deposits, for example of NPG.

In a very particularly preferred embodiment, the residence time of the condensed condensate before recycling into the column is sufficiently long that at least a portion of the NPG formate present in the condensate is cleaved to NPG and formic acid. Preferably 10 to 100% by weight, more preferably 40 to 90% by weight and especially preferably 50 to 80% by weight of the NPG formate present in the condensate is cleaved to NPG and formic acid. The residence time depends specifically on the temperature, the pH and the composition of the condensate. The higher or lower the pH and/or the higher the temperature of the distillate or the more or less water is present in the distillate, the shorter the residence time may be.

The pH of the condensate after condensation is preferably adjusted by adding pH regulators such that the pH of the condensate is within the desired range.

In a particularly preferred embodiment, the pH of the condensate is in the range from 7.1 to 14, preferably 8 to 12 and especially preferably 9 to 11. In a further preferred embodiment, the pH of the condensate is in the range from 1 to 6.9, preferably 2 to 5 and especially preferably 3 to 4. The pH regulator used is preferably one or more substances selected from the group consisting of tertiary amine, an inorganic base, an inorganic acid and an organic acid. The substances may be present in liquid form mixed homogeneously with the condensate, or heterogeneously in solid form.

The tertiary amines used may be amines as described, for example, in DE-A 28 13 201 and DE A 27 02 582. Particular preference is given to tri-n-alkylamines, especially triethylamine, tri-n-propylamine, tri-n-butylamine and trimethylamine. Very particular preference is given to trimethylamine (TMA), triethylamine (TEA) and tri-n-propylamine (TPA), since these compounds generally have a lower boiling point than the polymethylols formed with preference and hence the distillative removal from the reaction mixture is facilitated. Especially preferably, trimethylamine (TMA) is used as the tertiary amine in the reaction. Particularly advantageously, the tertiary amine used is the same tertiary amine which has already been used beforehand in the aldolization stage as the catalyst.

The inorganic bases used are preferably carbonates, hydrogencarbonates and hydroxides of the alkali metals and alkaline earth metals, more preferably Na₂CO₃, K₂CO₃, CaCO₃, NaHCO₃, KHCO₃, NaOH, KOH and Ca(OH)₂. Inorganic bases can be used as a solution, preferably as an aqueous solution, preferably in a concentration of 5 to 50% by weight.

The inorganic or organic acids may, in accordance with the invention, be mineral acids such as sulfuric acid or phosphoric acid, or organic acids such as citric acid, acetic acid, formic acid or ethylhexanoic acid. Preference is given to using formic acid.

As a heterogeneous pH regulator, a basic or acidic catalyst can be added to the system, for example an aluminosilicate doped with acid or basic elements, or an ion exchanger consisting of an organic skeleton and acidic or basic end groups.

Preferably, TMA or TEA is added to the condensate as a pH regulator.

In a further preferred embodiment, water is fed into the condensate from the first evaporator. Water can promote the cleavage of the NPG formate ester by hydrolysis. Preferably, a sufficient amount of water is fed into the condensate so that the weight ratio of water to NPG formate is in the range from 5:100 to 100:100, more preferably 10:100 to 50:100 and especially preferably 20:100 to 30:100. In a very particularly preferred embodiment, water is fed in from the condensate of the second condenser.

The temperature of the condensate is preferably below the condensation temperature of NPG formate at the appropriate column top pressure, and generally corresponds to the condenser temperature set.

It is preferable not to heat the condensate any further before recycling it into the column, or to heat it only to such an extent that it remains guaranteed that the condensate does not partially evaporate and remains essentially in the liquid phase. On the other hand, it is preferable that the condensate is not cooled any further, since the redissociation of NPG formate proceeds more rapidly at higher temperatures. It is therefore preferable that pipelines or delay vessels are correspondingly insulated. In order to accelerate the redissociation, the condensate can also be adjusted to a higher pressure after the condensation. This variant, however, is not preferred since the condensate has to be cooled down again before introduction into the columns, in order that it does not evaporate at the pressure employed in the column. The residence time can be adjusted by, for example, using pipelines of appropriate length between condenser and the column. The residence time can also be adjusted by introducing the condensate into a delay vessel. The residence time is generally determined by the ratio of feed stream and volume and the delay vessel. The delay vessel or the pipelines can optionally be heated, although the temperature should not exceed the temperature of the coolant at the inlet of the first condenser, in order that the condensate remains essentially liquid. The residence time between condensation and introduction into the column (recycling of the reflux) is preferably between 5 minutes and 300 minutes, more preferably 10 minutes to 240 minutes and especially preferably 15 minutes to 120 minutes.

The gas stream downstream of the first condenser possibly comprises fine droplets which may consist, for example, of water, NPG and NPG formate. In a preferred variant, these are removed from the gas stream with the aid of a droplet separator of appropriate dimensions, for example a cyclone, a structured packing, demister or depth filter, and passed into the condensate vessel, such that the loss of product of value in the process is minimized further.

From the first condenser or the droplet separator, the uncondensed portion of the stream drawn off from the column is passed into a second condenser. The second condenser is preferably operated such that the predominant amount of water and methanol in the stream fed in is condensed. The condenser is preferably operated at a temperature at which water and methanol condense at the appropriate column top pressure. Preferably 70 to 100% by weight, more preferably 80 to 100% by weight, most preferably 90 to 99% by weight, of the gaseous stream which is fed to the second condenser is condensed out in the second condenser. For this purpose, it is preferable that the second condenser is operated at a temperature which is 1 to 80%, more preferably 20 to 80% and most preferably 40 to 60% below the condensation temperature of water at the appropriate top pressure. In a particular embodiment, the top pressure of the column is in the range from 0.01 to 0.5 bar. At 0.01 bar, the condensation temperature of water is approximately 8° C.; at 0.5 bar, the condensation temperature of methanol is approx. 82° C. The result of this is that the temperature of the cooling medium at the inlet of the second condenser at 0.01 bar is in the range from 8° C.×0.2 to 8° C.×0.99, more preferably in the range from 8° C.×0.2 to 8° C.×0.8 and especially preferably in the range from 8° C.×0.4 to 8° C.×0.6. A further result of this is that the temperature of the cooling medium at the inlet of the second condenser at 0.5 bar is in the range from 82° C.×0.2 to 82° C.×0.99, more preferably in the range from 82° C.×0.2 to 82° C.×0.8 and especially preferably in the range from 82° C.×0.4 to 82° C.×0.6.

The condenser can be configured from virtually all condensers known to those skilled in the art, for example plate condensers, shell and tube condensers or coil condensers. The condenser is preferably configured as a shell and tube condenser. The cooling medium used here may preferably be very cold water (e.g. about 5° C.) or a coolant mixture (e.g. glycol-water at, for example, −20° C.).

The portion of the stream supplied to the second condenser which is not condensed and remains gaseous under these conditions, for example TMA, can be discharged from the process, for example by sending it to incineration. However, this gaseous stream can also be purified by distillation, such that recovered TMA can be recycled back into the process. The condensed stream from the second condenser, which comprises predominantly water and methanol, can be worked up by distillation or introduced directly into a waste water treatment.

In a further preferred embodiment, the condensers are connected in such a way that the cooling medium is conducted first into the second condenser and from the outlet of the second condenser to the inlet of the first condenser. Such a connection can achieve the effect that the cooling medium is first heated in the second condenser, such that it already has the aforementioned inlet temperature at the inlet of the first condenser. It is thus possible to achieve an efficient integrated energy and heat system between the condensers. The amount of coolant which is conducted from the outlet of the second condenser to the inlet of the first condenser is optionally reduced further by drawing off a portion of the amount of coolant between second and first condenser.

In a further preferred embodiment, a smaller amount of condensate is condensed out in the first condenser than is required as reflux for a sufficient separating performance in the distillation of the aqueous NPG. In this embodiment, the deficient amount of reflux is then made up from the condensate from the second condenser. This can achieve the effect that no more condensate is obtained in the first condenser than is required for the reflux and which would otherwise have to be discharged from the process or stored intermediately. The condensate from the second condenser can be introduced as reflux either separately or together with the condensate from the first condenser. When the condensate from the first condenser is to be introduced together with the condensate from the second condenser, it is preferable to undertake the mixing just before introduction into the column, since the lower temperature of the condensate from the second reactor could slow the redissociation of NPG formate.

Preference is given to discharging an output comprising predominantly NPG from the bottom of the evaporator. Discharge from the circulation stream of the evaporator is also possible. The bottom output is referred to in the context of the present invention as “crude NPG”. The crude NPG thus obtained comprises a small proportion of polymethylol formate. The proportion of polymethylol formate is preferably less than 1500 ppm by weight, preferably less than 1200 ppm by weight, more preferably less than 800 ppm by weight and especially preferably less than 600 ppm by weight.

The crude NPG preferably has the following composition:
90 to 99% by weight of NPG (I),
0.01 to 5% by weight hydroxypivalic acid,
0 to 5% by weight of organic secondary compounds.

The crude NPG more preferably has the following composition:
95 to 99% by weight of NPG,
0.1 to 2% by weight of hydroxypivalic acid,
0 to 3% by weight of organic secondary compounds.

In order to remove the relatively high-boiling acidic components present in the bottoms, especially hydroxypivalic acid, with low loss of NPG, the bottoms evaporator used in the distillation is preferably at least one evaporator with short residence time, for example a falling film evaporator with residue discharge, a thin film evaporator or helical tube evaporator. In a particular embodiment, the bottom of the column may be configured as a tapering bottom, in order to further reduce the residence time in the column bottom.

The distillation of the crude NPG is preferably performed under the following conditions:

Advantageously, the condensate obtained in the condenser is recycled into the distillation column (return stream) to an extent of more than 30% by weight, more preferably to an extent of more than 50% by weight. The condensate is preferably recycled into the top of the column.

The condenser is preferably operated at a temperature in the range from 50 to 180° C., preferably 130 to 160° C.

The cooling medium used here may preferably as far as possible be water, which at the same time evaporates.

The top pressure is preferably 0.001 to 0.9 bar, more preferably 0.01 to 0.5 bar and most preferably 0.02 to 0.4 bar. The vacuum is typically generated on the industrial scale by means of a steam ejector.

The bottom temperature is generally selected such that NPG is converted to the gas phase, while hydroxypivalic acid remains in the column bottom. Preference is given to establishing a bottom temperature which is 100 to 150%, preferably 105 to 140%, more preferably 110 to 130%, of the boiling temperature of the NPG. For example, in the case of preparation of NPG using TMA as the tertiary amine and a pressure at the top of the column of 150 mbar, preference is given to establishing a column bottom temperature of 150 to 200° C., more preferably of 160 to 190° C.

The bottom of the distillation column is preferably connected to at least one evaporator with short residence time.

The bottom of the distillation column and the evaporator with short residence time together constitute, by definition, the evaporation stage.

According to the disclosure, the residence time of the evaporation stage is calculated by dividing the volume of the liquid holdup in the hot part of the column (V_{holdup}) by the sum of return stream and feed volume flow of the column (V_{holdup}/(feed stream+return stream)), the liquid holdup in the hot part of the column (V_{holdup}) being calculated from the volume of the holdup of the column bottom (V_{holdup, bottom}) plus the volume of the holdup of the evaporator (V_{holdup, evaporator}) (V_{holdup}=V_{holdup, bottom}+V_{holdup, evaporator}). The residence time in the evaporation stage is advantageously less than 45 minutes, preferably less than 30 minutes, more preferably less than 15 minutes, especially preferably less than 10 minutes and most preferably less than 5 minutes. In general, it is preferred to select the residence time in the evaporation stage such that a shorter residence time is correspondingly established at higher bottom temperatures.

At a bottom temperature which is in the range from 130 to 150% of the boiling temperature of the NPG, the residence time in the evaporation stage is preferably 5 minutes and less, more preferably 4 minutes and less, and most preferably 3 minutes and less.

At a bottom temperature which is within the range from 120 to 130% of the boiling temperature of the NPG, the residence time in the evaporation stage is preferably 30 minutes and less, more preferably 15 minutes and less and most preferably 10 minutes and less, and especially preferably 5 minutes and less.

At a bottom temperature which is within the range from 100 to 120% of the boiling temperature of the NPG, the residence time in the evaporation stage is preferably 45 minutes and less, more preferably 30 minutes and less and most preferably 15 minutes and less, and especially preferably 10 minutes and less.

In a further particular embodiment, the evaporator with short residence time is connected to at least one further evaporator with short residence time.

The bottom of the distillation column and the evaporator with short residence time, in this preferred embodiment, by definition, together constitute the first evaporation stage. The further evaporator(s) with short residence time, by definition, form(s) the second or the (1+n)th (where n≧2) evaporation stage. The evaporator with short residence time is preferably connected to one further evaporator with short residence time (two-stage configuration). In this embodiment, the predominant portion of the energy needed for evaporation is usually introduced in the first evaporation stage. In the second evaporator stage, the higher temperature required for evaporation can then be achieved with a shorter residence time, such that the residence time in the second evaporation stage is shorter. The first stage is preferably configured as a falling film evaporator or helical tube evaporator. The second stage of this particular embodiment is preferably a falling film evaporator, helical tube evaporator or thin layer evaporator.

According to the disclosure, the residence time in the first evaporation stage is calculated by dividing the volume of the liquid holdup in the hot part of the column (V_{holdup}) by the sum of return stream and feed volume flow of the column (V_{holdup}/(feed stream+return stream)), the liquid holdup in the hot part of the column (V_{holdup}) being calculated from the volume of the holdup of the column bottom (V_{holdup, bottom}) plus the volume of the holdup of the evaporator (V_{holdup, evaporator}) (V_{holdup}=V_{holdup, bottom}+V_{holdup, evaporator}). According to the disclosure, the residence time of the second evaporation stage is calculated by dividing the liquid holdup of the second evaporator by the feed stream of the second evaporator.

According to the disclosure, the residence time of the (1+n)th evaporation stage is accordingly calculated by dividing the liquid holdup of the (1+n)th evaporator by the feed stream of the (1+n)th evaporator.

In this preferred embodiment, the bottom temperature in the first evaporation stage is advantageously above the evaporation temperature of the NPG. The bottom temperature in the first evaporation stage is preferably 100 to 130%, more preferably 110 to 125%, above the boiling temperature of the NPG. The temperature in the second evaporation stage is generally selected such that the NPG is converted virtually completely to the gas phase. The temperature in the second evaporation stage is preferably 105 to 150%, more preferably 120 to 150%, especially preferably 130 to 140%, above the boiling temperature of the NPG.

The residence time in the first evaporation stage is advantageously less than 45 minutes, preferably less than 30 minutes, more preferably less than 15 minutes, especially preferably less than 10 minutes and most preferably less than 5 minutes. The residence time in the second evaporation stage is advantageously less than 30 minutes, preferably less than 15 minutes, more preferably less than 5 minutes, especially preferably less than 2 minutes and most preferably less than 1 minute.

In general, it is preferred to select the residence time of the evaporation stage such that a shorter residence time is established correspondingly at higher bottom temperatures. As mentioned above, the evaporator with short residence time can be connected to more than one further evaporator with short residence time, for example to 2 or 3 evaporators, in which case the last of the evaporators in the chain constitutes the so-called last evaporation stage. The residence time and the temperatures in the last evaporation stage correspond to the residence times and temperatures of the second evaporation stage in the two-stage configuration.

In the preparation of NPG using TMA as the tertiary amine, in the first evaporation stage, a bottom temperature of 135 to 170° C., more preferably 150 to 160° C., can preferably be established at a residence time of less than 45 minutes, preferably less than 30 minutes. In the second evaporation stage, a temperature of 160 to 220° C., preferably 180 to 200° C., is preferably established at a residence time of less than 15 minutes, preferably less than 10 minutes and more preferably less than 5 minutes.

The distillation column preferably has internals for increasing the separating performance. The distillative internals may, for example, be present as a structured packing, for example as a sheet metal packing such as Mellapak 250 Y or Montz Pak, B1-250 type. It is also possible for a structured packing with relatively low or increased specific surface area to be present, or it is possible to use a fabric packing or a structured packing with another geometry such as Mellapak 252 Y. Advantages in the case of use of these distillative internals are the low pressure drop and the low specific liquid holdup compared to, for example, valve trays. The internals may be present in one or more sections.

The hydrogenation output is preferably fed in within a spatial region between ¼ and ¾ of the theoretical plates of the distillation column, more preferably within a spatial region between ⅓ and ⅔ of the theoretical plates of the distillation column. For example, the feed may be somewhat above the middle of the theoretical plates (ratio 3:4). The number of theoretical plates is generally in the range from 5 to 30, preferably 10 to 20.

Under these conditions, in general, NPG is removed from the higher-boiling hydroxypivalic acid.

In the condenser, purified NPG is preferably obtained as the condensate. The purity of the NPG is preferably at least 99.0% by weight, more preferably at least 99.2% by weight.

Preference is given to discharging, from the bottom of the evaporator, an output which comprises a predominantly higher-boiling compound, such as hydroxypivalic acid.

The bottoms can either be utilized thermally in an incineration or be fed to a downstream distillation column, by fractionating it into several fractions.

For example, the bottoms, in the case of preparation of NPG, can be fractionated into a low-boiling fraction, in particular containing hydroxypivalic acid, a medium-boiling fraction, in particular containing HPN (>97% HPN), and a high-boiling fraction (in particular esters of HPA and HPN).

The uncondensed residual vapors comprise generally, as well as leakage air and traces of water, predominantly NPG, and are advantageously recycled directly in gaseous form into distillation stage d).

Thus, the present application also provides a process for preparing neopentyl glycol, which comprises reacting isobutyraldehyde with formaldehyde in the presence of a tertiary amine and reacting the hydroxypivalaldehyde thus obtained either with hydrogen in the presence of catalysts or with formaldehyde under basic conditions, and purifying the aqueous NPG mixture thus obtained according to at least one of claims 1 to 13.

NPG is used principally as a component for the synthesis of polyester resins, unsaturated polyester resins, lubricants and plasticizers. The present invention accordingly also relates to a process for producing polyester resins, unsaturated polyester resins, lubricants or plasticizers, which comprises preparing NPG in a first stage in accordance with the invention and, in a second stage, using the NPG prepared in stage 1 for the production of polyester resins, unsaturated polyester resins, lubricants and plasticizers.

The advantages of the present invention are that it is possible by means of the process according to the invention to purify an aqueous NPG mixture, which can preferably originate from a two-stage hydrogenation process, such that NPG is obtained in high yield and high purity. The losses of NPG formate can be reduced, as a result of which the yield of NPG product of value can firstly be enhanced, but the costs for disposal of secondary streams can also be reduced. This makes the inventive NPG process particularly economically viable and environmentally friendly. A further advantage of the present invention is that the entire process for preparing NPG becomes more effective since the proportion of NPG lost in the purification can be reduced. Furthermore, it is possible to provide NPG with high purity, which is particularly suitable for further processing in the appropriate fields of use.

The invention is illustrated by the examples which follow:

### EXAMPLES

The examples which follow are based on simulation results which were achieved with the Chemasim™ software. The thermodynamic parameters used for the reactants, products and by-products in the program are based on published thermodynamic data or in-house measurements. In the case of the vapor/liquid equilibria, real binary substance behavior based on measurements or estimates was used for the mixtures of methanol, isobutanol, NPG, formic acid and TMA with water, and for the NPG/NPG formate and NPG/NPG isobutyrate mixtures. For the remaining mixtures, ideal behavior was assumed. The stated apparatuses used were specified and simulated with the customary routines present in the software. To optimize the simulation model, the simulated results were compared with experimental results, where available, and the simulation model was matched to the experimental results, such that a good agreement could be achieved between simulation and experimental data. The examples which follow were calculated with the optimized simulation model.

### Example 1

Preparation of an aqueous polymethylol mixture by the hydrogenation process

Stage a) Aldol reaction:

Approx. 750 g/h of isobutyraldehyde (approx. >99.5 GC area % of IBA) were reacted with approx. 700 g/h of formaldehyde (approx. 49% formaldehyde, 1.5% methanol, remainder water) and 80 g/h of trimethylamine solution (50% TMA in water) were reacted in a two-stage stirred tank cascade.

Stage b) Distillative separation of the reaction mixture from stage a):

Subsequently, the solution was freed of low boilers by distillation in a column. The column is equipped with 1.5 m of fabric packing (specific surface area 500 m²/m³) in the rectifying section and 4 m of sheet metal packing (250 m²/m³). The aldolization output was supplied above the sheet metal packing; at the top of the column, a condenser with cooling water (approx. 10° C.) and a downstream phase separator was used. At the top, the distillate was supplied to the condenser in gaseous form. Approx. 255 g/h of liquid condensate were obtained. In the downstream phase separator, an aqueous phase of 95 g/h was removed and supplied completely to the column. The phase separator also supplied 135 g/h to the first stirred tank. In order to maintain the regulation temperature at 85° C. in the column, 25 g/h of organic phase were additionally supplied to the column. In the cold trap downstream of the condenser, approx. 1 g/h of liquid was obtained (approx. 80% IBA, approx. 20% TMA), which had likewise been recycled.

The IBA removal was conducted at a top pressure of approx. 1 bar absolute. The evaporator used was a falling-film evaporator. A bottom temperature in the bottom of the column of 102° C. was established. The reflux rate (i.e. cooling water rate of the partial condenser) to the column was regulated by means of the temperature in the middle of the fabric packing; a temperature of 85° C. was established. From the bottom of the column, a pump was used to draw off approx. 100 kg/h of liquid. This was supplied to the falling-film evaporator (consisting of an oil-heated stainless steel tube, length 2.5 m, internal diameter approx. 21 mm, wall thickness approx. 2 mm). From the bottom of the falling-film evaporator, approx. 1.5 kg/h of product with a concentration of approx. 0.3% isobutyraldehyde were drawn off. The vapors and excess liquid were supplied to the column bottom. The bottom product discharged comprised approx. 70% by weight of HPA, approx. 1.5% by weight of HPN, 0.3% by weight of IBA, remainder water.

Stage c) Hydrogenation of the bottom output from stage b):

The bottom product obtained was subsequently subjected to a fixed bed hydrogenation. The catalyst was activated as follows: 150 ml of a Cu/Al203 catalyst as described in EP 44444 was activated in a tubular reactor at 190° C. by passing over a mixture of 5% by volume of hydrogen and 95% by volume of nitrogen (total volume 50 I (STP)/h) at ambient pressure for 24 h. The hydrogenation was conducted as follows: The starting solution used was the mixture described above as the hydrogenation feed. Approx. 10% by weight, based on the hydrogenation feed, of a 15% aqueous solution of trimethylamine was added to the mixture. The feed thus obtained was run through the reactor heated to 120° C. in trickle mode at H2 pressure 40 bar. The space velocity was 0.4 kg of HPA/(Icat.*h). A portion of the hydrogenation output was added again to the feed (circulation mode). The ratio of circulation to feed was 10:1. The pH of samples of the reactor output at room temperature was measured and found to be 8.9.

The composition of the aqueous polymethylol mixture from stage c) is:
NPG: 69% by weight
Methanol: 3.5% by weight
TMA: 2% by weight
Organic secondary compounds (HPS, isobutanol): <2% by weight
TMA formate: 1% by weight
Water: 23% by weight

### Example 2

Distillation of an aqueous polymethylol mixture (comparative example)

The following mixture was added (1.5 kg/h) at stage 9 to a distillation column having 30 theoretical plates and a plate efficiency of 36%:
NPG: 70% by weight
Methanol: 3% by weight
TMA: 0.7% by weight
Organic secondary compounds (HPS, NPG-IBT, isobutanol, etc.): <2% by weight
NPG formate: 0.4% by weight
Water: 24% by weight

The column was provided with a vaporizer in the bottom and with a simple condenser in the top. The top pressure was 200 mbar abs. and the bottom pressure 298 mbar abs. At the top, condensation was effected at 45° C. and the esters present there were hydrolyzed. The reflux ratio in the column was 0.45. At the bottom (approx. 171° C.), 1065 g/h were drawn off, which comprise 98.2% NPG. At the top, 428 g/h were drawn off, consisting of 84% water, 10% methanol and 2% NPG.

### Example 3

Distillation of an aqueous polymethylol mixture (inventive)

Under analogous conditions to ex. 2, a second condenser was added to the column. The reflux to the column was served only from the first (partial) condenser and the remaining vapor stream was condensed in the second condenser and discharged. This stream comprised 84% water, 10% methanol and only 0.1% NPG.

1-16. (canceled)

17. A process for distilling an aqueous NPG mixture comprising NPG, a tertiary amine, water and the adduct of tertiary amine and formic acid (amine formate), said distillation being performed in a distillation column, the process comprising:
drawing off a gaseous stream in the upper region of the column and feeding it to two condensers connected in series,
the first condenser being operated in such a way that a portion of the gaseous stream is condensed in the first condenser and
the second condenser being operated in such a way that the uncondensed portion of the gaseous stream is essentially fully condensed in the second condenser,
the condensed stream from the first condenser being recycled fully or partly as reflux into the distillation column.

18. The process according to claim 17, wherein the distillation column has a top pressure in the range from 0.001 to 0.9 bar.

19. The process according to claim 17, wherein the distillation column has a column bottom and the temperature in the column bottom is 5 to 50% above the boiling temperature of NPG formate at the particular column pressure.

20. The process according to claim 17, wherein 5 to 80% by weight of the gaseous stream fed to the first condenser is condensed out.

21. The process according to claim 17, wherein the second condenser has a coolant inlet and the temperature of coolant at the coolant inlet of the second condenser is −20 to 70° C.

22. The process according to claim 17, wherein the residence time of the condensed stream from the first condenser which is recycled into the column as reflux is sufficiently long that at least a portion of the NPG formate present in the condensate is cleaved to NPG and formic acid.

23. The process according to claim 17, wherein the residence time of the condensed stream from the first condenser which is recycled into the column as reflux is sufficiently long that 10 to 100% by weight of the NPG formate present in the condensate is cleaved to NPG and formic acid.

24. The process according to claim 17, wherein the pH of the condensate from the first condenser is adjusted by adding pH regulators such that the pH of the condensate is in the pH range from 7.1 to 14.

25. The process according to claim 17, wherein the pH of the condensate from the first condenser is adjusted by adding pH regulators such that the pH of the condensate is in the pH range from 1 to 6.9.

26. The process according to claim 17, wherein the amount of water in the condensate of the first condenser is adjusted such that the weight ratio of water to NPG formate is in the range from 100:0.1 to 100:100.

27. The process according to claim 17, wherein 70 to 100% of the gaseous stream which is fed to the second condenser is condensed out.

28. The process according to claim 17, wherein the second condenser comprises a coolant inlet and the temperature of the coolant at the coolant inlet of the second condenser is 1 to 80% below the condensation temperature of water at the particular column pressure.

29. The process according to claim 17, wherein the first and second condensers are connected in such a way that a cooling medium is conducted first into the second condenser and from the outlet of the second condenser to the inlet of the first condenser.

30. The process according to claim 17, wherein a smaller amount of condensate condensed out in the first condenser than is required as reflux for a sufficient separating performance in the distillation of the aqueous NPG, and the deficient amount of reflux for sufficient separating performance is made up from the condensate from the second condenser.

31. The process according to claim 17, wherein an apparatus for separation of droplets is mounted in the gas stream between the first and second condensers.

32. A process for preparing neopentyl glycol, which comprises reacting isobutyraldehyde with formaldehyde in the presence of a tertiary amine to obtain hydroxypivalinaldehyde and reacting the hydroxypivalinaldehyde thus obtained either with hydrogen in the presence of catalysts or with formaldehyde under basic conditions to obtain aqueous NPG mixture, and purifying the aqueous NPG mixture thus obtained by the process according to claim 17.

33. A process for producing polyester resins, unsaturated polyester resins, lubricants or plasticizers, which comprises preparing neopentyl glycol by the process according to claim 32 in stage 1 and, in a second stage, utilizing the neopentyl glycol prepared in stage 1 for the production of polyester resins, unsaturated polyester resins, lubricants and plasticizers.
